(19) United States
(12) Patent Application Publication
(10) Pub. No.: US 2022/0379076 A1
(43) Pub. Date: Dec. 1, 2022

(54) COVERED CATHETHER

(71) Applicant: Brian Jackson, Butler, AL (US)

(72) Inventor: Brian Jackson, Butler, AL (US)

(21) Appl. No.: 17/335,852

(22) Filed: Jun. 1, 2021

(51) Int. Cl. A61M 25/00; A61M 1/00

(52) U.S. Cl. CPC A61M 25/0043; A61M 1/84; A61M 2025/0062; A61M 2025/0079; A61M 2210/1085

(57) ABSTRACT

A covered catheter, including a catheter body coated with a lubricant to facilitate entry into tissue of a user, and a covering sheath, including a sheath body to cover at least a portion of the catheter body in an extended position, and expose the catheter body in a retracted position, a first end cap disposed on at least a portion of a first end of the sheath body to enclose the first end of the sheath body, and a second end cap disposed on at least a portion of a second end of the sheath body to enclose the second end of the sheath body.

### BACKGROUND

### 1. Field

The present general inventive concept relates generally to a catheter, and particularly, to a covered catheter.

### 2. Description of the Related Art

People who suffer from paralysis and/or restricted muscle function often require medical equipment to effectively alleviate themselves of waste. However, current medical tools are invasive and difficult to sanitize, which leads to potential discomfort and/or pain for those a patient who relies on Foley catheters.

Moreover, most catheters are left exposed to contaminants in an environment. In other words, the catheter will need to be cleaned before use because the sanitary condition of the catheter is likely unknown. As such, using the catheter without cleaning prior to use can expose the patient to infections.

Therefore, there is a need for a covered catheter that prevents a risk of contamination.

### SUMMARY

The present general inventive concept provides a covered catheter.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a covered catheter, including a catheter body coated with a lubricant to facilitate entry into tissue of a user, and a covering sheath, including a sheath body to cover at least a portion of the catheter body in an extended position, and expose the catheter body in a retracted position, a first end cap disposed on at least a portion of a first end of the sheath body to enclose the first end of the sheath body, and a second end cap disposed on at least a portion of a second end of the sheath body to enclose the second end of the sheath body.

The catheter body may include a first channel, and a second channel disposed within at least a portion of the first channel.

The catheter body may further include a bladder opening disposed on at least a portion of a first end of the catheter body and connected to the second channel to receive a liquid therein, a connection end disposed on at least a portion of a second end of the catheter body, the connection end including a balloon port connected to the first channel to receive a sterile liquid therein, and a drainage port connected to the second channel to expel the liquid received from the bladder opening, and a balloon disposed on at least a portion of the catheter body and connected to the first channel to expand in response to the balloon receiving the sterile liquid from the balloon port.

The sheath body may at least partially deform in response to moving the first end cap toward the second end cap.

The first end cap may expose the catheter body through a first end aperture in response to moving the first end cap toward the second end cap.

The first end cap may be movable and the second end cap is immovable.

The covering sheath may further include a clip disposed on at least a portion of an inner surface of the first end cap to detachably connect the first end cap to the second end cap.

The covered catheter may further include an antistatic agent disposed on at least a portion of the catheter body to prevent static electricity from building up on the catheter body.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a side perspective view of a covered catheter with a covering sheath in an extended position, according to an exemplary embodiment of the present general inventive concept;

FIG. 2 illustrates a side perspective view of the covered catheter with the covering sheath in a retracted position, according to an exemplary embodiment of the present general inventive concept;

FIG. 3A illustrates a sectional view of a catheter body, according to an exemplary embodiment of the present general inventive concept; and

FIG. 3B illustrates a sectional view of a catheter body, according to another exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Covered Catheter 100
Catheter Body 110
First Channel 110a
Second Channel 110b
Bladder Opening 111
Connection End 112
Balloon Port 112a
Port Valve 112a1
Drainage Port 112b
Balloon 113
Covering Sheath 120
Sheath Body 121
First End Cap 122
First End Aperture 122a
Clip 123
Second End Cap 124
Second End Aperture 124a
Antistatic Agent 130
Catheter Body 210
First Channel 210a
Second Channel 210b

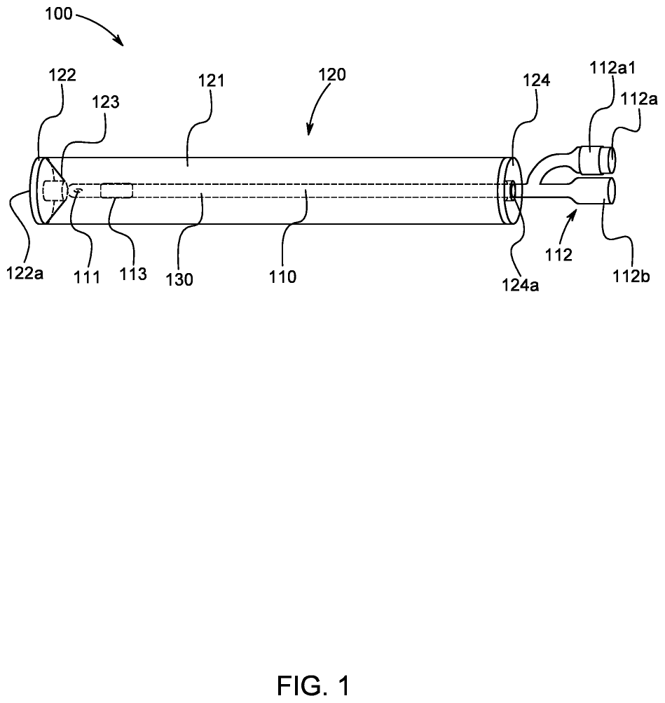

FIG. 1 illustrates a side perspective view of a covered catheter 100 with a covering sheath 120 in an extended position, according to an exemplary embodiment of the present general inventive concept.

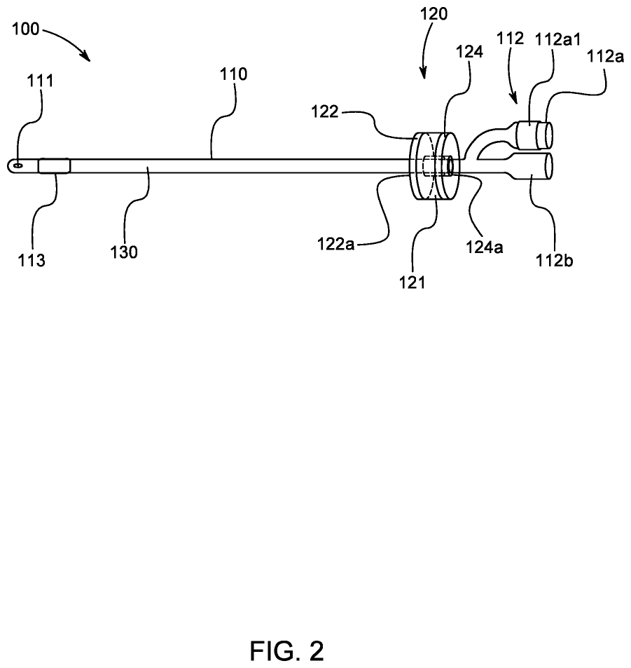

FIG. 2 illustrates a side perspective view of the covered catheter 100 with the covering sheath 120 in a retracted position, according to an exemplary embodiment of the present general inventive concept.

The covered catheter 100 may be constructed from at least one of plastic, metal, and rubber, etc., but is not limited thereto.

The covered catheter 100 may include a catheter body 110, a covering sheath 120, and an antistatic agent 130, but is not limited thereto.

Referring to FIG. 1, the catheter body 110 is illustrated to be a Foley catheter. However, the catheter body 110 may be a urinary catheter, a pigtail catheter, an angioplasty catheter, an intravenous catheter, a Swan-Ganz catheter, an embryo transfer catheter, and a Quinton catheter, but is not limited thereto. As such, the catheter body 110 may be applied in different applications of use, such as requiring medical treatment and/or surgical procedures.

The catheter body 110 may include a bladder opening 111, a connection end 112, and a balloon 113, but is not limited thereto.

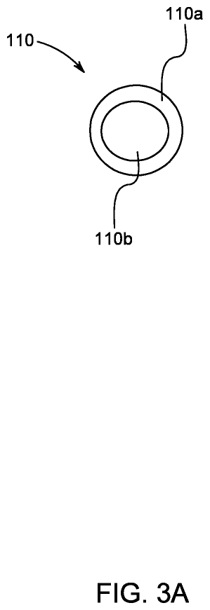

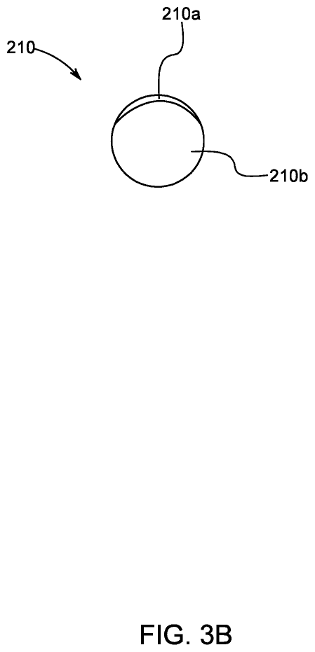

FIG. 3A illustrates a sectional view of a catheter body 110, according to an exemplary embodiment of the present general inventive concept.

FIG. 3B illustrates a sectional view of a catheter body 210, according to another exemplary embodiment of the present general inventive concept.

The catheter body 110 may further include a first channel 110a and a second channel 110b. The first channel 110a of the catheter body 110 may be separated from the second channel 110b, such that a first liquid in the first channel 110a may not contact and/or mix with a second liquid in the second channel 110b. The first channel 110a and/or the second channel 110b of the catheter body 110 may have a length less than and/or equivalent to a full length of the catheter body 110.

Referring to FIG. 3A, the first channel 110a may have a diameter and/or a circumference greater than a diameter and/or a circumference of the second channel 110b. Moreover, the second channel 110b maybe disposed within at least a portion of the first channel 110a.

Referring to FIG. 3B, alternatively, the catheter body 210 may include a first channel 210a and a second channel 210b. The first channel 210a may be disposed on at least a portion of the catheter body 210, such that the first channel 210a has a crescent shape. Moreover, the first channel 210a may have a size (i.e. volume) less than a size of the second channel 210b.

It is important to note that the catheter body 210 may include all other features described herein with respect to the catheter body 110 except the size and/or shape of the first channel 210a and/or the second channel 210b.

The catheter body 110 may be elongated and have a predetermined length based on the application of use. Moreover, the catheter body 110 may have a cylindrical and/or tubular shape. The catheter body 110 may be rigid and/or flexible on different portions thereof, depending on the application of use. For example, the catheter body 110 may resist bending at a first end and/or at least partially deform (i.e. bend, stretch, expand) in response to an application of force on a second end. Optionally, the catheter body 110 may return to its original shape in absence of the application of force.

The catheter body 110 may be coated with a lubricant to facilitate entry into tissue of a user and/or a patient. Furthermore, the lubricant may contain an anti-pathogenic agent therein. Therefore, the lubricant may prevent growth and/or eliminate at least one pathogen (e.g., bacteria, virus, parasite, fungus) on the catheter body 110.

The bladder opening 111 may be disposed on at least a portion of a first end of the catheter body 110 and connected to the second channel 110b and/or the second channel 210b. The bladder opening 111 may receive a liquid therein, such as urine. More specifically, the bladder opening 111 may receive the liquid in response to inserting the bladder opening 111 and/or the catheter body 110 into a urinary tract of the user and/or the patient.

The connection end 112 may include a balloon port 112a and a drainage port 112b, but is not limited thereto.

The connection end 112 may be disposed on at least a portion of a second end of the catheter body 110.

The balloon port 112a may receive a first external connection thereto. More specifically, the balloon port 112a may connect to a source of sterile liquid (e.g., sterile water). Additionally, the balloon port 112a may connect to the first channel 110a and/or the first channel 210a.

The balloon port 112a may include a port valve 112a1, but is not limited thereto.

The port valve 112a1 may be circumferentially disposed around at least a portion of the balloon port 112a. In other words, the port valve 112a1 may be disposed around a perimeter of the balloon port 112a in a circular shape. The port valve 112a1 may allow (i.e. unlock) movement of the sterile liquid into the balloon port 112a in response to rotating the port valve 112a1 in a first direction. Subsequently, the port valve 112a1 may prevent (i.e. lock) movement of the sterile liquid into the balloon port 112a in response to rotating the port valve 112a1 in a second direction. As such, the port valve 112a1 may control flow of the sterile liquid into the balloon port 112a.

The drainage port 112b may receive a second external connection thereto. More specifically, the drainage port 112b may connect to a collection container. Additionally, the drainage port 112b may connect to the second channel 110b and/or the second channel 210b. As such, the drainage port 112b may expel the liquid received within the bladder opening 111, such that the collection container stores the liquid therein. For example, the drainage port 112b may expel the urine into the collection container.

The balloon 113 may be disposed on at least a portion of the catheter body 110. Moreover, the balloon 113 may be constructed of different material than the catheter body 110. More specifically, the balloon 113 may be a thin rubber construction, such that the balloon 113 may increase (i.e. expand) in size in response to the balloon port 112a receiving the sterile liquid therein. In other words, the balloon 113 may appear to inflate in size. Conversely, the balloon 113 may decrease (i.e. shrink) in size in response to the sterile liquid within the first channel 110a and/or the first channel 210a being extracted from the balloon port 112a (e.g., via an external pump connected to the balloon port 112a). Therefore, the balloon 113 may prevent the first end of the catheter body 110 from exiting the tissue of the user and/or the patient after expanding within the tissue.

The covering sheath 120 may include a sheath body 121, a first end cap 122, a clip 123, and a second end cap 124, but is not limited thereto.

The sheath body 121 may be constructed of plastic, cloth, and/or rubber, but is not limited thereto.

The sheath body 121 may have a length extending at least a portion of the full length of the catheter body 110. The sheath body 121 may cover at least a portion of the catheter body 110. Referring again to FIG. 1, the sheath body 121 may extend to cover the catheter body 110 in a first lateral position. Referring to FIG. 2, the sheath body 121 may at least partially retract and/or collapse to expose the catheter body 110 in a second lateral position, such that the catheter body 110 may be at least partially inserted into the user and/or the patient. Accordingly, the sheath body 121 may at least partially deform (i.e. bend, expand, collapse) in response to an application of force thereto. Also, the sheath body 121 may prevent at least one particle (e.g., dirt, dust, liquid, virus, bacteria, parasite, fungus) from moving therethrough. Therefore, the sheath body 121 may be non-porous and/or may protect the catheter body 110 from contamination.

Also, the sheath body 121 may store the lubricant therein to coat the catheter body 110.

The first end cap 122 may include a first end aperture 122a, but is not limited thereto.

The first end cap 122 may be disposed on at least a portion of a first end of the sheath body 121. The first end cap 122 may prevent the at least one particle from moving therethrough. Also, the first end cap 122 may enclose the first end of the sheath body 121.

The first end aperture 122a may receive the catheter body 110 therethrough. More specifically, moving the first end cap 122 toward the second end cap 124 may at least partially deform the sheath body 121, such that the catheter body 110 may be exposed. Optionally, the first end aperture 122a may be covered by a light film, such that the catheter body 110 may puncture the light film in response to moving through the first end aperture 122a.

The clip 123 may be disposed on at least a portion of an inner surface of the first end cap 122.

The second end cap 124 may include a second end aperture 124a, but is not limited thereto.

The second end cap 124 may be disposed on at least a portion of a second end of the sheath body 121. The second end cap 124 may prevent the at least one particle from moving therethrough. Also, the second end cap 124 may enclose the second end of the sheath body 121. As such, the second end cap 124 may prevent skin irritation of the user and/or the patient during use.

The second end aperture 124a may receive the catheter body 110 therethrough. However, the second end aperture 124a may remain at a predetermined location of the catheter body 110. In other words, the second end cap 124 may be immovable on the catheter body 110 unlike the first end cap 122. Additionally, the second end aperture 124a may receive the clip 123 therein in response to moving the first end cap 122 toward the second end cap 124, such that the clip 123 may detachably connect to the second end cap 124. As such, the clip 123 may snap into the second end aperture 124a.

The antistatic agent 130 may include aliphatic amines, quaternary ammonium salts, polyethylene glycol esters, and/or an ionic liquid, but is not limited thereto.

The antistatic agent 130 may be disposed on at least a portion of the catheter body 110. The antistatic agent 130 may prevent static electricity from building up on the catheter body 110.

Therefore, the covered catheter 100 may facilitate insertion into the tissue of the user and/or the patient. Also, the covered catheter 100 may prevent contamination due to being covered.

The present general inventive concept may include a covered catheter 100, including a catheter body 110 coated with a lubricant to facilitate entry into tissue of a user, and a covering sheath 120, including a sheath body 121 to cover at least a portion of the catheter body 110 in an extended position, and expose the catheter body 110 in a retracted position, a first end cap 122 disposed on at least a portion of a first end of the sheath body 121 to enclose the first end of the sheath body 121, and a second end cap 124 disposed on at least a portion of a second end of the sheath body 121 to enclose the second end of the sheath body 121.

The catheter body 110 may include a first channel 110a, and a second channel 110b disposed within at least a portion of the first channel 110a.

The catheter body 110 may further include a bladder opening 111 disposed on at least a portion of a first end of the catheter body 110 and connected to the second channel 110b to receive a liquid therein, a connection end 112 disposed on at least a portion of a second end of the catheter body 110, the connection end 112 including a balloon port 112a connected to the first channel 110a to receive a sterile liquid therein, and a drainage port 112b connected to the second channel 110b to expel the liquid received from the bladder opening 111, and a balloon 113 disposed on at least a portion of the catheter body 110 and connected to the first channel 110a to expand in response to the balloon 113 receiving the sterile liquid from the balloon port 112a.

The sheath body 121 may at least partially deform in response to moving the first end cap 122 toward the second end cap 124.

The first end cap 122 may expose the catheter body 110 through a first end aperture 122a in response to moving the first end cap 122 toward the second end cap 124.

The first end cap 122 may be movable and the second end cap 124 is immovable.

The covering sheath 120 may further include a clip 123 disposed on at least a portion of an inner surface of the first end cap 122 to detachably connect the first end cap 122 to the second end cap 124.

The covered catheter 100 may further include an antistatic agent 130 disposed on at least a portion of the catheter body 110 to prevent static electricity from building up on the catheter body 110.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A covered catheter, comprising:
a catheter body coated with a lubricant to facilitate entry into tissue of a user; and
a covering sheath, comprising:
a sheath body to cover at least a portion of the catheter body in an extended position, and expose the catheter body in a retracted position,
a first end cap disposed on at least a portion of a first end of the sheath body to enclose the first end of the sheath body, and prevent access within the sheath body while the sheath body is in the extended position and expose access within the sheath body while the sheath body is in the retracted position, and
a second end cap disposed on at least a portion of a second end of the sheath body to enclose the second end of the sheath body.

2. The covered catheter of claim 1, wherein the catheter body comprises:
a first channel; and
a second channel disposed within at least a portion of the first channel.

3. The covered catheter of claim 2, wherein the catheter body further comprises:
a bladder opening disposed on at least a portion of a first end of the catheter body and connected to the second channel to receive a liquid therein;
a connection end disposed on at least a portion of a second end of the catheter body, the connection end comprising:
a balloon port connected to the first channel to receive a sterile liquid therein, and
a drainage port connected to the second channel to expel the liquid received from the bladder opening; and
a balloon disposed on at least a portion of the catheter body and connected to the first channel to expand in response to the balloon receiving the sterile liquid from the balloon port.

4. The covered catheter of claim 1, wherein the sheath body at least partially deforms in response to moving the first end cap toward the second end cap.

5. The covered catheter of claim 1, wherein the first end cap exposes the catheter body through a first end aperture in response to moving the first end cap toward the second end cap.

6. The covered catheter of claim 1, wherein the first end cap is movable and the second end cap is immovable.

7. The covered catheter of claim 1, wherein the covering sheath further comprises:
a clip disposed on at least a portion of an inner surface of the first end cap to detachably connect the first end cap to the second end cap.

8. The covered catheter of claim 1, further comprising:
an antistatic agent disposed on at least a portion of the catheter body to prevent static electricity from building up on the catheter body.

9. A covered catheter, comprising:
a catheter body coated with a lubricant to facilitate entry into tissue of a user; and
a covering sheath, comprising:
a sheath body to cover at least a portion of the catheter body in an extended position, and expose the catheter body in a retracted position,
a first end cap disposed on at least a portion of a first end of the sheath body to enclose the first end of the sheath body and protrude toward a second end of the sheath body within the sheath body, and
a second end cap disposed on at least a portion of a second end of the sheath body to enclose the second end of the sheath body.
